(19) United States
(12) Patent Application Publication
(10) Pub. No.: US 2008/0311207 A1
(43) Pub. Date: Dec. 18, 2008

(54) Micelles and Nanoemulsions for Preventive and Reactive Treatment of Atherosclerosis

(76) Inventors: Manoj Varshney, Gainesville, FL (US); Dinesh O. Shah, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/662,157

(22) PCT Filed: Sep. 8, 2005
(86) PCT No.: PCT/US05/31840

§ 371 Date: Mar. 31, 2008

Related U.S. Application Data
(60) Provisional application No. 60/608,347, filed on Sep. 8, 2004.

(51) Int. Cl. A61K 9/14

(52) U.S. Cl. 424/489; 977/773

(57) ABSTRACT

The subject invention is directed to microemulsion-based (ME) nanoparticles and methods of using same. The ME nanoparticles of the subject invention encompass self-assemblies of oil in water emulsions in the presence of at least two emulsifiers. One of the emulsifiers is a salt of a fatty acid, and the combined concentration of the at least two emulsifiers is sufficiently large to produce micelles, wherein the oil droplets are the hydrophobic core of the micelles. The subject invention also contemplates methods of modifying lipids, high density lipoprotein (HDL), and low density lipoprotein (LDL) in blood by contacting the blood with the ME nanoparticles of the subject invention. Another aspect concerns methods for treating atherosclerosis by administering the ME nanoparticles of the subject invention to a patient in need thereof.

### CROSS-REFERENCE TO A RELATED APPLICATION

This application claims the benefit of U.S. provisional application Ser. No. 60/608,347, filed Sep. 8, 2004, which is hereby incorporated by reference in its entirety including all figures, tables, and drawings.

The subject matter of this application has been supported in part by U.S. Government Support under National Science Foundation Grant No. EEC-94-02989. Accordingly, the U.S. Government has certain rights in this invention.

### BACKGROUND OF THE INVENTION

The subject invention relates to novel microemulsion-based nanoparticles and methods for the prevention and treatment of atherosclerosis by the administration of these nanoparticles. The subject invention also relates to methods for modifying the concentration of blood lipids, high density lipoproteins (HDL), and low density lipoproteins (LDL).

Atherosclerosis is a condition where plaque, which is a combination of fatty deposits, calcium, blood components, cells, and cholesterol, builds up on the inner walls of arteries throughout the body. As the plaque buildup increases, the affected artery or arteries narrows resulting in decreased blood flow through the affected area. As a result, atherosclerosis is a leading cause of cardiovascular disease. Although the causes of atherosclerosis are still under investigation, three different contributing factors have been identified in the buildup of plaque including arterial wall damage, inflammation, and high cholesterol levels. Symptoms of atherosclerosis typically present after one or more arteries are sufficiently blocked with plaque so that blood flow is significantly reduced possibly producing pain or discomfort. Unfortunately, in many persons, no symptoms present until an artery is completely blocked, often by a blood clot in a narrow artery, thereby causing a heart attack or stroke. Accordingly, there is a need to prevent or reduce the level of atherosclerosis even before symptoms present. One such way is to monitor and modify the cholesterol levels in the blood.

50% of Americans have levels of cholesterol that increase their risk of developing atherosclerosis. Cholesterol itself is a necessary fatty substance naturally made by the liver and used to produce hormones, vitamin D, and bile acids. However, dietary factors contribute to the high levels of cholesterol found in many Americans. Accordingly, the medical community recommends monitoring the total cholesterol, HDL, LDL, and triglyceride levels for persons over twenty years of age.

HDL is a beneficial lipoprotein that is colloquially referred to as “good cholesterol.” Although not a cholesterol, it does circulate in the blood stream and serves as a transportation mechanism to deliver cholesterol to the hepatobiliary system, specifically the liver. It extracts cholesterol from tissues and converts it into hydrophobic esters. A low concentration of HDL (<40 mg/dL) is considered a risk factor in the development of cardiovascular disease.

LDL is a lipoprotein that is often called “bad cholesterol.” A high concentration of LDL can contribute to the formation of plaque on arterial walls. A LDL cholesterol level of 130-159 mg/dL is considered borderline high. A level of 160-189 mg/dL is considered high, and a LDL level above 190 mg/dL is considered very high.

Though many drugs are available commercially for controlling cholesterol levels and reducing atherosclerosis-related events, no method exists for active treatment of atherosclerosis. Current treatments for cholesterol levels are either invasive or indirect. Lifestyle modifications like regular exercise, proper nutrition, and smoking cessation are known to reduce risk factors associated with atherosclerosis. Various cholesterol-lowering drugs are highly prescribed, including statins like locastatin, pravastatin, simvastatin, and atorvastatin and bile acid sequesterants like cholestruramine and colestipol. Other medications that can be administered to reduce LDL levels are gemfibrozil, clofibrate, and probucol.

New medicaments are under development that mimic that mechanisms of HDL in the blood. Synthetic versions of a genetic variant of Apolipoprotein A-1 (ApoA-1) are under investigation that appear to prevent lipid oxidation, thereby preventing cholesterol deposits in artery walls. It has been shown that administration of a hydrophobic surfactant, poloxalene 2930, as a dietary supplement affects the HDL and LDL levels in rabbit lipoproteins (Rodgers, J. B. et al. “Hydrophobic Surfactant Treatment Prevents Atherosclerosis in the Rabbit.” The Journal of Clinical Investigation. 1983; 71: 1490-94).

Currently, however, administration of these medicaments and improvements in lifestyle have not been sufficient to prevent accumulation of plaque on arterial wall surfaces and elevated cholesterol levels in some persons. As a result, the next phase of treatment of atherosclerosis involves invasive, mechanical repairs. One option for mechanical repair is coronary angioplasty, which increases blood flow by inserting a catheter to create a bigger opening in a blood vessel. Although angioplasty is performed in other blood vessels, percutaneous transluminal coronary angioplasty (PTCA) refers to angioplasty in the coronary arteries to permit more blood flow into the heart.

Balloon angioplasty uses a small balloon that is inflated inside the blocked artery to open the blocked area. Atherectomy refers to shaving away the blocked area inside the artery by using a tiny device on the end of a catheter. In laser angioplasty, a laser is used to “vaporize” the blockage in the artery.

A tiny coil is expanded inside the blocked artery to open the blocked area and is left in place to keep the artery open in a coronary artery stent procedure. Brachytherapy refers to a type of radiation therapy in which gamma or beta radioactive materials are placed in direct contact with the tissue being treated with the goal of suppressing restenosis following angioplasty.

Coronary artery bypass grafting (CAGB) is an invasive, yet common, surgery wherein a portion of a healthy blood vessel is grafted around a blocked artery to restore blood flow. Although angioplasty and CAGB are common treatments for atherosclerosis, they are not cures, and the disease can continue without addressing the underlying causative factors.

### BRIEF SUMMARY OF THE SUBJECT INVENTION

The subject invention provides materials and methods for the prevention and treatment of atherosclerosis and other disorders. Furthermore, the lipid levels of blood are modified by contacting microemulsion-based (ME) nanoparticles of the subject invention with blood, thereby increasing the concentration of HDL, decreasing the concentration of LDL, and/or decreasing the concentration of triglycerides. Advantageously, the subject invention provides non-invasive treatment options.

The ME nanoparticles of the subject invention relate to self-assemblies of oil in water in the presence of at least two emulsifiers wherein one of the emulsifiers is a salt of a fatty acid. The concentration of the at least two emulsifiers is sufficient to form micelles so that oil droplets form the hydrophobic core of the nanoparticles within the emulsion as illustrated in FIG. 5A. Advantageously, the ME nanoparticles of the subject invention can be further modified to attach drugs or nutrient supplements to the surface of the nanoparticle or, in the case of hydrophobic or lipophilic drugs or nutrient supplements, partition them into the core of the ME nanoparticles.

The subject invention also is directed to methods for modifying the concentrations of lipids, HDL, and LDL in blood by contacting blood with the ME nanoparticles of the subject invention. In a specific embodiment, the methods first comprise identifying a patient with at least one risk factor associated with atherosclerosis.

In yet another aspect, the subject invention relates to methods for treating atherosclerosis by administering the ME nanoparticles of the subject invention to a patient in need thereof. The administration step encompasses all manners of routes including orally, intranasally, intra-arterially, and intramuscular. The preferred administration route is parenterally via intravenous administration. This aspect of the subject invention may optionally comprise the simultaneous or sequential administration of drugs, nutrient supplements, or combination of both.

### BRIEF DESCRIPTION OF THE DRAWINGS

FIG. 1 illustrates a graphical comparison of heparinized human blood samples (n=5) that were incubated with 0.001%_{v/v}, 0.003%_{v/v}, and 0.01%_{v/v} ME615 nanoemulsion. The HDL concentration was determined enzymatically based on the Trinder procedure using a BIOSCANNE2000 device and PTS test strips. To compensate for the dilution effect, an equal amount of phosphate buffered saline was added to the control samples.

FIG. 2 shows a graphical illustration of the HDL concentration (mmol per liter) of two blood samples obtained from LDLR knockout mice. The nanoemulsion ME615 was injected into one sample in a 10%_{v/v} ME615 to blood volume ratio. The concentrations were measured one hour later.

FIG. 3A illustrates a transmission electron microscopic image of an aorta from a normal mouse.

FIG. 3B illustrates a transmission electron microscopic image of an aorta from a mouse fed a LDLR-null high cholesterol diet and administered four doses of ME615. Note the break in cell membrane of the foam cell and extrusion of the intracellular component.

FIG. 4A illustrates the particle volume distribution of human serum particles as a function of particle size.

FIG. 4B illustrates the size dispersion of ME615.

FIG. 4C illustrates the size dispersion of human serum particles treated with 10%_{v/v} ME615.

FIG. 4D illustrates the size distribution of human serum treated with 20%_{v/v} ME615.

FIG. 5A illustrates a schematic of an oil-in-water nanoemulsion. The darker shaded area indicates an oil droplet surrounded by water (designated with the lighter shaded area). A plurality of surfactants is shown at the interface between the oil and the water.

FIG. 5B is a photograph detailing the differences in the optical qualities of an emulsion to a nanoemulsion in accordance with the subject invention. The particle size within the emulsion is ˜400 nm, which a surface area of ˜15 m²/ml. In contrast, the transparent nanoemulsion contains nanoparticles having a particle size of ˜30 nm with a far greater surface area of ˜215 m²/ml.

### DETAILED DISCLOSURE OF THE SUBJECT INVENTION

The subject invention provides materials and methods to treat and prevent atherosclerosis in patients. One aspect of the subject invention pertains to microemulsion-based (ME) nanoparticles. The ME nanoparticles of the subject invention comprise an oil in water nanoemulsion prepared from the self-assembly of a biocompatible, oil in water in the presence of at least two emulsifiers, wherein one emulsifier is a salt of a fatty acid.

More specifically, an oil-in-water nanoemulsion exists if a small amount of oil is dispersed in a large amount of water in the presence of an emulsifier under the appropriate conditions. The solution may optionally be agitated to resolve turbidity of the solution.

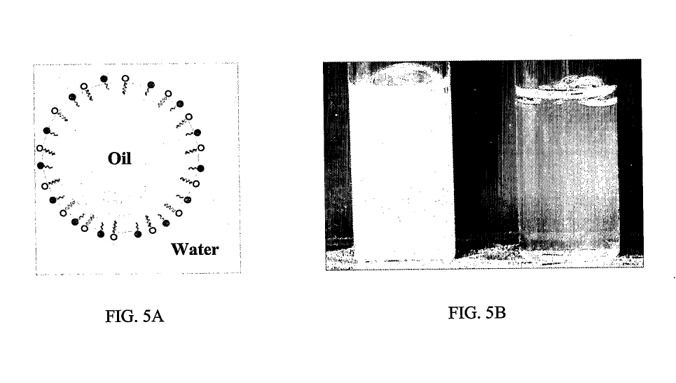

Advantageously, the diameter of the resulting ME nanoparticles is within the range of about 10 nm to about 120 nm. The transparency resulting from this range of nanoparticle diameter size is illustrated in FIG. 5B. Preferably, the ME nanoparticle range is between about 10 nm to about 60 nm. More preferably, the ME nanoparticle size range is within 10 nm to about 40 nm. Most preferably the ME nanoparticle size is about 30 nm. ME nanoparticles within this size can penetrate cellular walls and, for some size nanoparticles, nuclei. Advantageously, nanoparticle sizes less than 60 nm in diameter have extended life in circulation due to reduced uptake by the reticuloendothelial system (Allemann, E. et al. Eur. J. Pharm. Biopharm. 1993; 39: 173).

The concentration of the at least two emulsifiers is sufficiently large to form micelles. As is known in the art, micelles aggregate at concentrations equal to or greater than the critical micelle concentration (CMC) into a roughly spherical shape. There is a critical concentration below which a surfactant will not form micelles. The amphiphilic emulsifiers self-assemble in water with the hydrophobic tails radially arranged surrounding oil droplets and forming a hydrophobic core. The hydrophilic heads form the surface of the nanoparticle seeking maximum exposure to water (see FIG. 5A).

Surfactants (or emulsifiers) are classified according to the ionic type of the hydrophilic group, ionic or non-ionic. Ionic surfactants generally have a lower CMC than non-ionic emulsifiers, and they provide low particle size emulsions.

Non-ionic and ionic surfactants utilized in the subject ME nanoparticles can include, without limitation, lauryl alcohol (+6EO); nonyl phenol (+10EO, +15EO, +30EO); sodium lauryl sulphate; lauryl sulphate (+2EO, +4EO) Na salt; sodium dodecylbenzene sulphonate; sodium dioctyl sulphosuccinate; polyvinyl alcohol; polyol; unsaturated and/or saturated sodium or potassium salts of fatty acids; and all synthetically modified PEG surfactants.

Preferred nonionic surfactants are poloxamers, symmetric triblock copolymers of ethylene oxide (EO) and propylene oxide (PO), for example the PLURONIC line of surfactants (BASF Corporation, Florham Park, N.J.), denoted by the formula HO-(EO)ₓ(PO)_{y}(EO)ₓ-H where x and y each indicate the number of units of EO and PO, respectively, the —OH substituent represents a hydroxyl group and H represents hydrogen. In certain embodiments of the subject invention, x is within the range of 6 to 104, and y is within the range of 20 to 65. Numerous surfactants in the PLURONIC family are available commercially. Preferred molecular weights of a poloxamer utilized in the subject invention varies between about 900 and about 14,600 with the weight fraction of the EO block ranging between about 0.1 to about 0.8.

Some exemplary triblock copolymers include HO-EO₁₀₀PO₆₅EO₁₀₀-H (i.e., PLURONIC F127), HO-EO₇₈PO₃₀EO₇₈-H (i.e., PLURONIC F68), HO-EO₁₁PO₂₀EO₁₁-H (i.e., PLURONIC L44), HO-EO₆PO₃₅EO₆-H (i.e., PLURONIC L62), HO-EO₁₃PO₃₀EO₁₃-H (i.e., PLURONIC L64), HO-EO₅₃PO₃₈EO₅₃-H (i.e., PLURONIC F77), HO-EO₅₉PO₄₃EO₅₉-H (i.e., PLURONIC F87), HO-EO₁₀₄PO₃₉EO₁₀₄-H (i.e., PLURONIC F88), and HO-EO₂₇PO₆₁EO₂₇-H (i.e., PLURONIC P104). Other suitable poloxamers include PLURONIC 10R5, PLURONIC 17R2, PLURONIC 17R4, PLURONIC 25R2, PLURONIC 25R4, PLURONIC 31R1, PLURONIC F108, PLURONIC F38, PLURONIC F87, PLURONIC F98, PLURONIC L10, PLURONIC L101, PLURONIC L121, PLURONIC L31, PLURONIC L35, PLURONIC L43, PLURONIC L61, PLURONIC L62D, PLURONIC L81, PLURONIC L92, PLURONIC N-3, PLURONIC P103, PLURONIC P105, PLURONIC P105, PLURONIC P123, PLURONIC P65, PLURONIC P84, and PLURONIC P85. Advantageously, the triblock copolymers aggregate when exposed to water due to its amphiphilic nature (i.e., the more hydrophilic EO sandwiches the less hydrophilic PO).

The salts of the fatty acids utilized in the ME nanoparticles of the subject invention can include, without limitation, non-toxic sodium or potassium salts of C₆ to C₂₀ fatty acids having the formula R(CO)O⁻M⁺, wherein R is a terminal hydrocarbon chain, and M⁺ is a positive ion. Salts utilized in other specific embodiments of the subject ME nanoparticles include salts of hexadecanoic acid; salts of octadecanoic acid; salts of 9-octadecanoic acid; salts of 9,12-octadecanoic acid; or salts of 9,12,15-octadecanoic acid. Preferably, the terminal hydrocarbon chain lengths of the fatty acid salts can be C₈ to C₁₆. More preferably, the salt is sodium caprylate.

Any biocompatible oil can be used to assemble the microemulsions of the subject invention. Exemplary biocompatible oils include, without limitation, vegetable oils (e.g., soy bean, olive, corn, safflower, coconut, canola, sesame, peanut, cotton seed, palm, myglol, and rape seed oils), eicosapentaenoic acid oil, propylene glycol, and acid esters of glycerol. More preferably, the oil is ethylbutyrate. The water is generally provided by a normal saline solution (0.9% NaCl_{w/v}) so that the resulting emulsion is isotonic.

The concentrations of the individual components of the ME nanoparticles of the subject invention encompass about eight parts of the emulsifier that is a salt of a fatty acid and about one part of any other emulsifier as discussed elsewhere to about 9 parts of oil in a isotropic normal saline solution. The concentration of fatty acid salt can vary between about 10 mM to about 190 mM whereas the concentration of any other emulsifier varies from about 4 mM to about 12 mM. The biocompatible oil concentration varies from about 20 mM to about 250 mM. A specific embodiment of a ME nanoparticle of the subject invention comprises about 8 mM of a poloxamer surfactant (e.g., HO-EO₁₀₀PO₆₅EO₁₀₀-H or PLURONIC F127), about 48 mM of a salt of fatty acid (e.g., sodium caprylate), and about 150 mM of a biocompatible oil (e.g., ethylbutyrate).

Optionally, the MEs of the subject invention can further comprise drugs, nutrient supplements, or combinations of both. For example, drugs can include those that work synergistically with the subject invention to treat and prevent atherosclerosis, lower LDL concentrations, and/or increase HDL concentrations. Exemplary drugs include aspirin, policosanol, beta-blockers (e.g., altenolol, metaprolal, nadolol, propranolol), calcium channel-blockers (e.g., diltiazem, nifedipine, verapamil), angiotensin-converting enzyme inhibitors (e.g., captopril, enalaprihn lisinopril), angiotension II receptor blockers (e.g., losartan, losartan in combination with hydrochlorthizide, olmesartan), statins (e.g., lovastatin, prevastatin, simvastatin, atorvastatin, atorvastatin), bile acid sequesterants (e.g., cholestyramine, colestipol, gemifibrozil, clofibrate, probucol), anti-inflammatories, antibiotics, and their pharmaceutically acceptable salts.

Exemplary nutrient supplements include choline, folic acid, B-6, B-12, niacin, niacin and chromium in combination, vitamin C, vitamin E, coenzyme Q10, and omega-3 oils. In one specific embodiment, at least one drug or nutrient supplement is partitioned into the hydrophobic core containing the biocompatible oil droplets of the subject ME nanoparticles. Advantageously, the hydrophobic core of the ME nanoparticles may be designed to carry drugs that are immiscible in aqueous solutions. In yet another embodiment, at least one drug or medicament is attached to the surface of the subject ME nanoparticle by a linker. Exemplary linkers include, without limitation, carboxylic esters, carboxamides, polylactides, carbohydrates, or any other biocompatible moiety useful for attaching a drug or nutrient supplement to the surface of ME nanoparticles of the subject invention.

Yet another aspect of the subject invention relates to methods for contacting a plurality of ME nanoparticles of the subject invention with human or non-human animal blood. The contacting step encompasses blood in vivo or in vitro. In one specific embodiment, the contacting step encompasses removing blood from a patient, contacting the removed blood with the subject ME nanoparticles, and then returning the blood to that patient (e.g., in a dialysis center). In yet another embodiment, the contacting step comprises removing blood from a blood donor, contacting the donated blood with a plurality of ME nanoparticles of the subject invention, and storing the treated blood for subsequent donation to a patient in need of a blood transfer. In yet another specific embodiment, the contacting step is applicable to research applications in vitro or within animal models.

In a preferred embodiment of the subject invention, the concentrations of lipids, LDL, and HDL are modified by contacting the blood (or tissue) bearing cholesterol with ME nanoparticles of the subject invention. HDL concentrations increase significantly following administration of the subject ME nanoparticles. The concentrations of LDL in blood also decrease. Without being limited by theory, the ME nanoparticles of the subject invention may modulate the surface property of apolipoprotein to accelerate the self-assembly of HDL, to block the cholesterol ester transfer protein (CETP) enzyme that is responsible for the transfer of HDL to low density lipoproteins (LDL), to partition lipophilic cholesterol and triglyceride molecules into the hydrophobic core of the ME nanoparticles, or a combination of any of the foregoing.

This aspect of the subject invention optionally first comprises identifying a patient having at least one risk factor associated with atherosclerosis and contacting the ME nanoparticles with the patient's blood. These risk factors include, without limitation, elevated C-reactive protein levels in blood (greater than 3 mg/L), elevated total cholesterol levels (greater than 200 mg/dL), elevated LDL levels (greater than 130 mg/dL), depressed HDL levels (less than 40 mg/dL), elevated triglyceride levels (greater than 150 mg/dL), a history of smoking or exposure to second hand smoke, high blood pressure (greater than 140 over 90 mm Hg), high fat diet, diabetes, physically inactive, overweight, ongoing stress, men over 45 years of age, menopausal or post-menopausal women, family history of heart attack or stroke before age 65, family history of angina, family history of high cholesterol or blood pressure, personal history of heart attack or stroke, and pre-menopausal women who smoke and take birth control pills.

The subject invention also pertains to methods for treating arthrosclerosis by administering ME nanoparticles of the subject invention to a patient in need thereof. A patient in need thereof can be one experiencing one or more of the following risk factors: elevated C-reactive protein levels in blood (greater than 3 mg/L), elevated total cholesterol levels (greater than 200 mg/dL), elevated LDL levels (greater than 130 mg/dL), depressed HDL levels (less than 40 mg/dL), elevated triglyceride levels (greater than 150 mg/dL), a history of smoking or exposure to second hand smoke, high blood pressure (greater than 140 over 90 mm Hg), high fat diet, diabetes, physically inactive, overweight, ongoing stress, men over 45 years of age, menopausal or post-menopausal women, family history of heart attack or stroke before age 65, family history of angina, family history of high cholesterol or blood pressure, personal history of heart attack or stroke, and pre-menopausal women who smoke and take birth control pills.

Methods of administration include, but are not limited to, intra-arterial, intramuscular, intravenous, intranasal, and oral routes. In a specific embodiment, the pharmaceutical compositions of the invention can be administered locally to the area in need of treatment; such local administration can be achieved, for example, by local infusion during surgery, by injection, by dialysis, or by means of a catheter.

Therapeutic amounts can be readily determined and will vary with the extent of atherosclerosis in the subject being treated, the concentrations of HDL, LDL, and lipids including triglycerides, the subject being treated, and the efficacy and toxicity of the ME nanoparticles. Similarly, suitable dosage formulations and methods of administering the ME nanoparticles can be readily determined by those of skill in the art.

The subject nanoparticles can be administered by any of a variety of routes, such as orally, intranasally, parenterally or by inhalation therapy, and can take form of tablets, lozenges, granules, capsules, pills, ampoule, suppositories or aerosol form. They can also take the form of suspensions, solutions, and emulsions of the active ingredient in aqueous or non-aqueous diluents, syrups, granulates or powders. In addition to an agent of the present invention, the subject nanoparticles can also contain other pharmaceutically active compounds. Formulations are described in a number of sources that are well known and readily available to those skilled in the art. For example, Remington's Pharmaceutical Sciences (Martin E W, 1995, Easton Pa., Mack Publishing Company, 19^{th} Ed.) describes formulations that can be used in connection with the subject invention.

Aqueous solutions of nanoparticles are most conveniently used. Administration may be achieved by any route or method. In a preferred administration, the ME nanoparticles (and compositions comprising the nanoparticles) can be administered parenterally, such as by intravenous administration.

Formulations suitable for parenteral administration include, for example, aqueous sterile injection solutions (0.9% NaCl_{w/v}), which may contain antioxidants, buffers, bacteriostats, and solutes that render the formulation isotonic with the blood of the intended recipient; and aqueous and non-aqueous sterile suspensions that may include suspending agents and thickening agents.

The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

In yet another embodiment, the dosage form for the ME nanoparticles are oral forms, for example, hard shell capsules, tablets, or coated capsules and tablets. The preferred oral dosage form is a capsule. Suitable capsules can be prepared from, for example, gelatin or hydroxypropylmethyl cellulose (HPMC). In this formulation, the capsule contain the ME nanoparticles. The capsule can further include at least one coating layer. One layer may be an enteric layer that prevents the capsule from decomposing in the acidic environment of the stomach. Other coatings may be barrier layers, semipermeable layers, expandable layers, and the like as known to one skilled in the art and discussed in, for example, U.S. Pat. No. 6,929,803.

In one specific embodiment, the ME nanoparticles can be formulated as nanogels, i.e., hydrogel based nanoparticles. Hydrogels are polymer networks that advantageously swell when exposed to water and are hydrophilic. (Peppas et al., “Hydrogels in pharmaceutical formulations,” Eur. J. Pharm. Biopharm. 50: 27-46 (2000).) Hydrogels can be formulated to have a low viscosity at room temperature or during administration but also form a hydrogel after ingestion because of the increase in temperature.

Nanogel formulations of the present invention can be in forms other than particulates. For example, a liquid or otherwise non-particulate hydrogel formulation may exhibit improved pharmacokinetics when exposed to the conditions of the stomach and GI tract.

Advantageously, the subject nanoparticles can be administered simultaneously or sequentially with other drugs or nutrient supplements. Examples include, but are not limited to, aspirin, policosanol, beta-blockers (e.g., altenolol, metaprolal, nadolol, propranolol), calcium channel-blockers (e.g., diltiazem, nifedipine, verapamil), angiotensin-converting enzyme inhibitors (e.g., captopril, enalaprilm lisinopril), angiotension II receptor blockers (e.g., losartan, losartan in combination with hydrochlorthizide, olmesartan), statin (e.g., lovastatin, prevastatin, simvastatin, atorvastatin, atorvastatin), bile acid sequesterants (e.g., cholestyramine, colestipol, gemifibrozil, clofibrate, probucol), anti-inflammatory agents, antibiotics, and their pharmaceutically acceptable salts. Exemplary nutrient supplements include choline, folic acid, B-6, B-12, niacin, niacin and chromium in combination, vitamin C, vitamin E, coenzyme Q10, and omega-3 oils.

In one embodiment, simultaneous administration of a drug or nutrient supplement is accomplished in the specific embodiment where at least one drug or nutrient supplement is partitioned into the hydrophobic core of the ME-based nanoparticles of the subject invention; in yet another embodiment, at least one drug or nutrient supplement is attached to the surface of the ME-based nanoparticles. In another embodiment, the ME nanoparticles of the invention can be associated with an implantable or deployable medical device. In a specific device, a stent implanted in a coronary artery to prop open an artery that has recently been treated with angioplasty can be coated with a microemulsion containing the subject nanoparticles. Optionally, the device may release the nanoparticles in a controlled fashion.

In one embodiment of the methods, pharmaceutical compositions of the nanoemulsions are administered to human or non-human animals to relieve a thrombosis of an artery. Advantageously, this specific embodiment is not limited to coronary thrombosis only but can also be applied therapeutically to treat strokes or post-operative thrombus or embolism in an emergent or non-emergent situation. Accordingly, the subject invention can be used to reduce the number of high-risk surgeries by treating a life-threatening illness medically rather than surgically.

In yet another aspect, the subject invention is directed to methods for the administration of nanoparticles, which are prepared in accordance with the subject invention, to a human or non-human animal in a pharmaceutically effective amount.

The subject invention also pertains to methods for preparing the ME nanoparticles. Advantageously, the ME nanoparticles are self-assemblies of oil in water. Accordingly, the methods of preparing the ME nanoparticles include contacting a biocompatible oil with water in the presence of a sufficient concentration of at least two emulsifiers so that micelles form, wherein one of the emulsifiers is a salt of a fatty acid. Optionally, an agitating step can resolve any turbidity in the emulsion.

As used in this specification the singular “a”, “an”, and “the” include plural reference unless the contact dictates otherwise. Thus, for example, a reference to “a nanoparticle” includes more than one such nanoparticle. A reference to “a micelle” includes more than one such micelle. A reference to “a cell” includes more than one such cell. A reference to “a targeting agent” includes more than one such targeting agent.

As used herein, an “effective amount” of ME nanoparticles or nanoemulsions or microemulsions or micelle is that amount effective to bring about the physiological changed desired in the biosystem to which the nanoparticles are administered.

The term “pharmaceutically effective amount” as used herein, means that amount of MEs, alone or in combination with another agent according to the particular aspect of the invention, that elicits the biological or medicinal response in a biosystem that is being sought by a researcher, veterinarian, medical doctor or other clinician, which includes alleviation of the symptoms of the disease or disorder being treated.

The terms “nanoparticle”, “nanosphere”, and “micelle” are used interchangeably to refer to a roughly spherical shaped unit that self-assembles under the appropriate conditions from an amphiphilic material so that the core is hydrophobic and the corona is hydrophilic.

As used herein, the term “drug” is interchangeable with the term “bioaffecting agent” and refers to any agent, including nutrition supplements, vitamins, minerals, and herbs, capable of having a physiologic effect (e.g., a therapeutic or prophylactic effect) on a biosystem. For the purposes of this application the physiologic effect may include lowering the total cholesterol level, lowering the LDL concentration, increasing the HDL concentration, decreasing any inflammation in the vascular system or arterial walls, preventing lipid oxidation, thinning the blood, treating any bacteria infections, treating any yeast or fungal growth, and any effects associated with administering beta blockers, calcium channel blockers, angiotensin II receptor blockers, statins, bile acid sequesterants, aspirin, policosanol, choline, folic acid, B-6, B-12, niacin, niacin and chromium in combination, vitamin C, vitamin E, coenzyme Q10, and omega-3 oils.

As used herein, the terms “biosystem”, “host”, “patient”, “recipient”, and “subject” are used interchangeable and, for the purposes of the subject invention, include both blood and blood products and tissues that bear cholesterol, LDL, and/or HDL. MEs of the subject invention may be administered to such targets in vitro or in vivo. Thus, the methods of administration are applicable to both human therapy and veterinary applications, as well as research applications in vitro or within animal models.

As used herein, the term “biocompatible” is interchangeable with non-toxic and refers to the ME nanoparticles of the subject invention that are innocuous when used in appropriate amounts and under appropriate conditions in the various administration routes contemplated by the subject invention's methods of administration.

The terms “comprising”, “consisting of”, and “consisting essentially of” are defined according to their standard meaning and may be substituted for one another throughout the instant application in order to attach the specific meaning associated with each term.

Following are examples, which illustrate procedures for practicing the invention. These examples should not be construed as limiting. All percentages are by weight and all solvent mixture proportions are by volume unless otherwise noted.

### EXAMPLE 1

Heparinized human blood samples were incubated with the nanoemulsion designated ME615, and total cholesterol, triglycerides (TG), high density lipoprotein-cholesterol (HDL-C), and low density lipoprotein-cholesterol (LDL-C) concentrations were monitored. ME615 refers to ME nanoparticles containing the following concentrations and components: 8 mM PLURONIC F127, 48 mM sodium caprylate, and 150 mM ethylbutyrate in normal saline. To compensate for the dilution effect, an equal amount of phosphate buffered saline (PBS) was added to the control samples. The lipid panel was determined enzymatically based on the Trinder procedure using a Bioscanner2000 device and PTS test strips, while LDL-C was determined by a direct method. The effective particle size and polydispersity (i.e., distribution width) of the nanoemulsion treated and untreated serum samples were measured after filtering thorough a 20 nm filter by the dynamic light scattering method using a submicron particle size analyzer (90Plus, Brookhaven Instruments Corporation, Holtsville, N.Y.). This instrument measures particle sizes that range from about 2 to 3000 nM in any liquid.

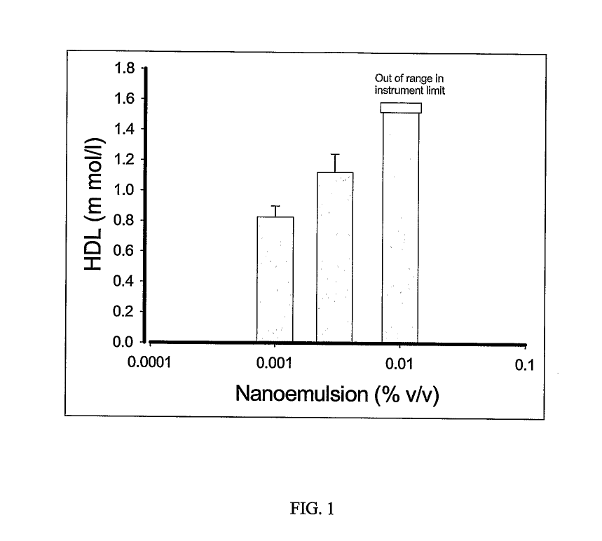

ME615 reduced LDL-C by 25-35% (n=5) and more importantly raised HDL-C 3-fold (Table 1). In control experiments, there was no cross-reactivity of the nanoemulsions with lipid measurements. Further, the increase in HDL-C was dependent on the concentration of the nanoemulsion and the incubation time (n=5, P<0.01) (FIG. 1). The increase in HDL-C became evident as early as 30 min after incubation. These effects were persistent throughout the period of observation.

TABLE 1
Effect of 10% v/v of ME615 nanoemulsion (NE) on lipids.

| Lipid Panel | Control (mM) | NE (mM/L) |
|---|---|---|
| Cholesterol, Total | 4.10 ± 0.25 | 3.55 ± 0.40 |
| Triglyceride | 2.52 ± 0.42 | 1.96 ± 0.28 |
| HDL-C | 0.78 ± 0.12 | >2.10 ± 0.04* |
| Direct LDL | 2.35 ± 0.27 | 1.63 ± 0.16* |

*P < 0.005

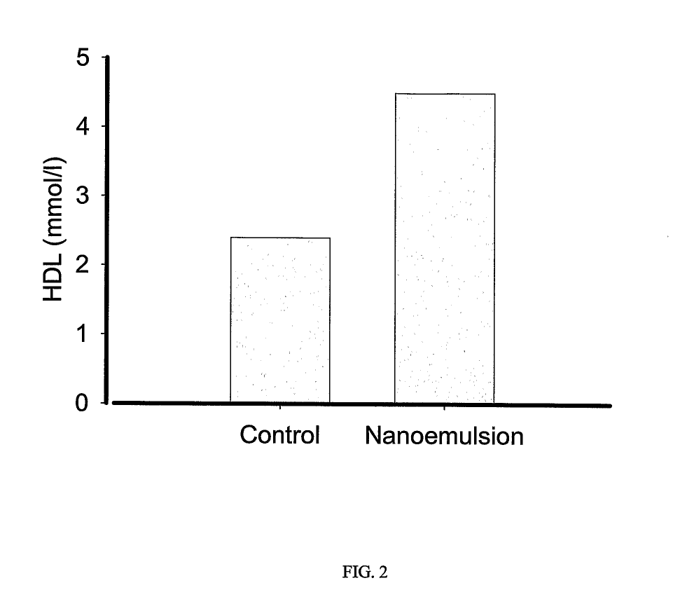

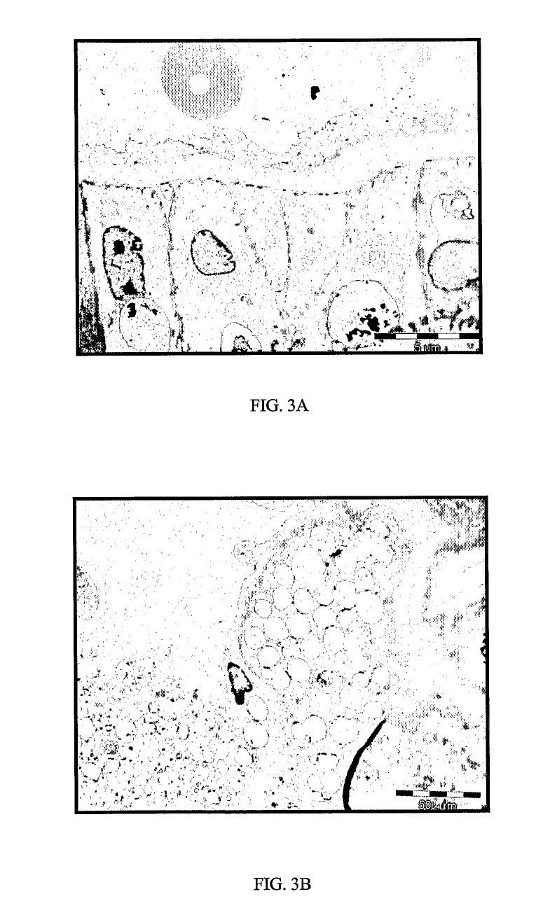

A similar, highly significant, increase in HDL-C upon addition of nanoemulsions was also observed in blood from the LDLR knockout mice (FIG. 2). The precise mechanism for these dramatic changes in blood lipids, particularly HDL-C, is not known, but it is possible that both LDL-C and TG molecules being lipophilic partition into the hydrophobic core of nanoemulsion, thereby reducing their free concentration in blood, while the HDL-C concentration increases. The anti-atherosclerotic effect of nanoemulsion was secondary to a decline in LDL-C and TG and a massive rise in HDL-C; one LDLR null mouse (treated with 2% high cholesterol diet) was treated with 4 doses of nanoemulsion. The transmission electron microscopic images of the aorta showed that nanoemulsion administration appeared to cause break down of foam cells with extrusion of cytosolic fatty deposits (FIGS. 3A and 3B).

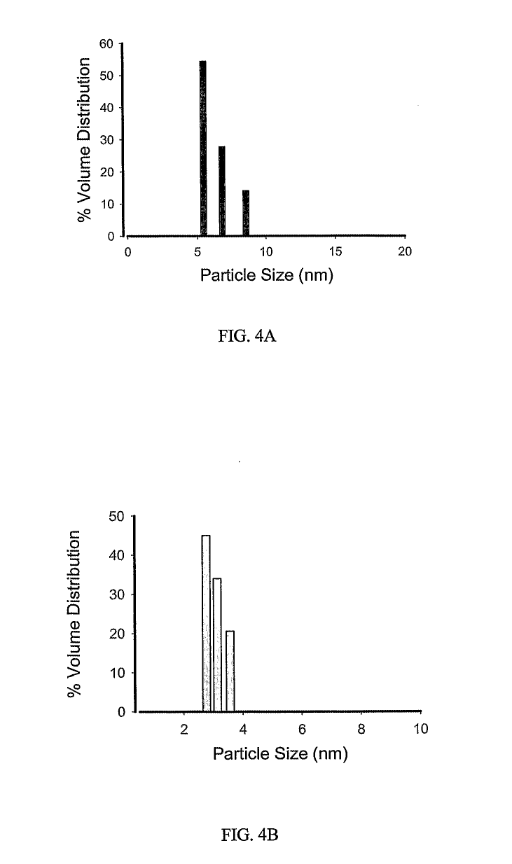

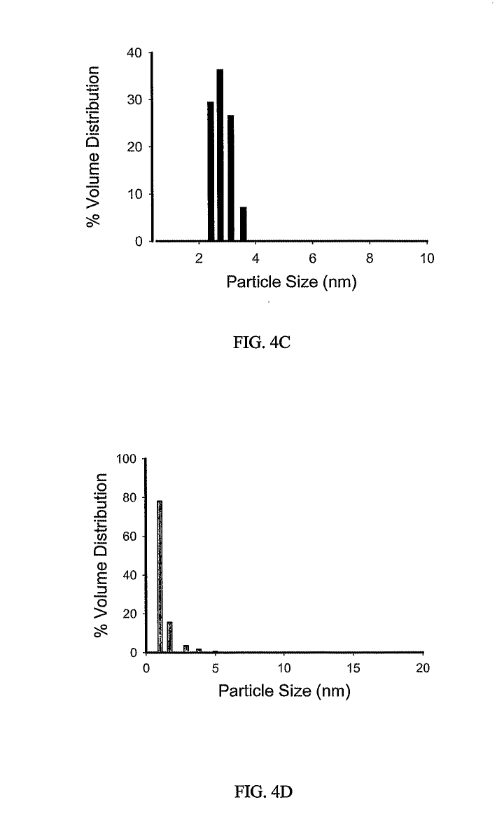

Particle size measurements of nanoemulsion treated serum depicts that the untreated serum particles ranges from 5-9 nm and are reduced to 3 nm in size after treatment with the 10% and 20% v/v nanoemulsions (FIGS. 4A-4D).

The results indicate that ME615 interacts with the blood lipids and reduces the LDL-C level in human blood. In LDLR knockout mice, the administration of ME615 addresses their lipid abnormalities. However, a detailed study on molecular level is needed to find out the lipid-nanoemulsion interaction mechanism.

All patents, patent applications, provisional applications, and publications referred to or cited herein are incorporated by reference in their entirety to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment (thereof disclosed herein can be combined with any and/or all other elements or limitations (individually or in any combination) or any other invention or embodiment thereof disclosed herein, and all such combinations are contemplated with the scope of the invention without limitation thereto.

1. A microemulsion-based nanoparticle comprising a self-assembly of a biocompatible oil in water in the presence of a sufficient concentration of at least two emulsifiers so that a plurality of micelles is formed, each micelle having a hydrophobic core and a hydrophilic surface, one emulsifier being a salt of a fatty acid.

2. The microemulsion-based nanoparticle according to claim 1, further comprising a drug, a nutrient supplement, or combination of both attached to the surface of the microemulsion-based nanoparticle or within the hydrophobic core of the microemulsion-based nanoparticle.

3-4. (canceled)

5. The microemulsion-based nanoparticle according to claim 1, wherein the biocompatible oil comprises ethylbutyrate.

6. The microemulsion-based nanoparticle according to claim 1, wherein one of the at least two emulsifiers is a poloxamer.

7. The microemulsion-based nanoparticle according to claim 1, wherein one of the at least two emulsifiers is a poloxamer comprising a symmetric triblock of ethylene oxide and propylene oxide, wherein the weight fraction of the ethylene oxide is between about 0.1 to about 0.8, and the molecular weight of the poloxamer is between about 900 and about 14,600.

8. The microemulsion-based nanoparticle according to claim 1, wherein one of the at least two emulsifiers is selected from the group consisting of HO-EO₁₀₀PO₆₅EO₁₀₀-H, HO-EO₇₈PO₃₀EO₇₈-H, HO-EO₁₁PO₂₀EO₁₁-H, HO-EO₆PO₃₅EO₆-H, HO-EO₁₃PO₃₀EO₁₃-H, HO-EO₅₃PO₃₈EO₅₃-H, HO-EO₅₉PO₄₃EO₅₉-H, HO-EO₁₀₄PO₃₉EO₁₀₄-H, and HO-EO₂₇PO₆₁EO₂₇-H, wherein
EO=ethylene oxide and
PO=propylene oxide.

9-13. (canceled)

14. The microemulsion-based nanoparticle according to claim 1, wherein the biocompatible oil is ethylbutyrate in a concentration of about 20 mM to about 250 mM, the salt of a fatty acid is sodium caprylate in a concentration of about 10 mM to about 190 mM, the at least two emulsifiers other than the salt of a fatty acid in a concentration of about 4 mM to about 12 mM, and the water is normal saline.

15. (canceled)

16. A method for modifying the concentrations of lipids, HDL, and LDL in blood comprising contacting blood with a composition comprising a plurality of microemulsion-based nanoparticles of claim 1.

17-20. (canceled)

21. The method according to claim 16, wherein the biocompatible oil comprises ethylbutyrate.

22. The method according to claim 16, wherein one of the at least two emulsifiers is a poloxamer.

23. The method according to claim 16, wherein one of the at least two emulsifiers is a poloxamer comprising a symmetric triblock of ethylene oxide and propylene oxide, wherein the weight fraction of the ethylene oxide is between about 0.1 to about 0.8, and the molecular weight of the poloxamer is between about 900 and about 14,600.

24. The method according to claim 16, wherein one of the at least two emulsifiers is selected from the group consisting of HO-EO₁₀₀PO₆₅EO₁₀₀-H, HO-EO₇₈PO₃₀EO₇₈-H, HO-EO₁₁PO₂₀EO₁₁-H, HO-EO₆PO₃₅EO₆-H, HO-EO₁₃PO₃₀EO₁₃-H, HO-EO₅₃PO₃₈EO₅₃-H, HO-EO₅₉PO₄₃EO₅₉-H, HO-EO₁₀₄PO₃₉EO₁₀₄-H, and HO-EO₂₇PO₆₁EO₂₇-H, wherein
EO=ethylene oxide and
PO=propylene oxide.

25-29. (canceled)

30. The method according to claim 16, wherein the biocompatible oil is ethylbutyrate in a concentration of about 20 mM to about 250 mM, the salt of a fatty acid is sodium caprylate in a concentration of about 10 mM to about 190 mM, the at least two emulsifiers other than the salt of a fatty acid in a concentration of about 4 mM to about 12 mM, and the water is normal saline.

31. The method according to claim 16, wherein the biocompatible oil is ethylbutyrate in a concentration of about 150 mM, the salt of a fatty acid is sodium caprylate in a concentration of about 48 mM, one of the at least two emulsifiers is HO-EO₁₀₀PO₆₅BE₁₀₀-H in a concentration of about 8 mM, and the water is normal saline.

32. A method of treating atherosclerosis comprising administering to a patient in need thereof an effective amount of a microemulsion-based nanoparticle of claim 1.

33-34. (canceled)

35. The method according to claim 32, wherein the biocompatible oil comprises ethylbutyrate.

36. The method according to claim 32, wherein one of the at least two emulsifiers is a poloxamer.

37. The method according to claim 32, wherein one of the at least two emulsifiers is a poloxamer comprising a symmetric triblock of ethylene oxide and propylene oxide, wherein the weight fraction of the ethylene oxide is between about 0.1 to about 0.8, and the molecular weight of the poloxamer is between about 900 and about 14,600.

38. The method according to claim 32, wherein one of the at least two emulsifiers is selected from the group consisting of HO-EO₁₀₀PO₆₅EO₁₀₀-H, HO-EO₇₈PO₃₀EO₇₈-H, HO-EO₁₁P₂₀EO₁₁-H, HO-EO₆PO₃₅EO₆-H, HO-EO₁₃PO₃₀EO₁₃-H, HO-EO₅₃PO₃₈EO₅₃-H, HO-EO₅₉PO₄₃EO₅₉-H, HO-EO₁₀₄PO₃₉EO₁₀₄-H, and HO-EO₂₇PO₆₁EO₂₇-H, wherein
EO=ethylene oxide and
PO=propylene oxide.

39-43. (canceled)

44. The method according to claim 32, wherein the biocompatible oil is ethylbutyrate in a concentration of about 20 mM to about 250 mM, the salt of a fatty acid is sodium caprylate in a concentration of about 10 mM to about 190 mM, the at least two emulsifiers other than the salt of a fatty acid in a concentration of about 4 mM to about 12 mM, and the water is normal saline.

45. (canceled)

46. The method according to claim 32, wherein the administration step comprises parenteral or oral administration.

47. (canceled)

48. A method for preparing a microemulsion-based nanoparticle of claim 1, wherein the method comprises contacting biocompatible oil with water in the presence of a sufficient concentration of at least two emulsifiers so that a plurality of micelles is formed, each micelle having a hydrophobic core and a hydrophilic surface, one emulsifier being a salt of a fatty acid.

49. The method according to claim 48, further comprising attaching a drug, a nutrient supplement, or combination of both to the surface of the microemulsion-based nanoparticle or within the hydrophobic core of the microemulsion-based nanoparticle.

50-51. (canceled)

52. The method according to claim 48, wherein the biocompatible oil comprises ethylbutyrate.

53. The method according to claim 48, wherein one of the at least two emulsifiers is a poloxamer.

54. The method according to claim 48, wherein one of the at least two emulsifiers is a poloxamer comprising a symmetric triblock of ethylene oxide and propylene oxide, wherein the weight fraction of the ethylene oxide is between about 0.1 to about 0.8, and the molecular weight of the poloxamer is between about 900 and about 14,600.

55. The method according to claim 48, wherein one of the at least two emulsifiers is selected from the group consisting of HO-EO₁₀₀PO₆₅EO₁₀₀-H, HO-EO₇₈PO₃₀EO₇₈-H, HO-EO₁₁PO₂₀EO₁₁-H, HO-EO₆PO₃₅EO₆-H, HO-EO₁₃PO₃₀EO₁₃-H, HO-EO₅₃PO₃₈EO₅₃-H, HO-EO₅₉PO₄₃EO₅₉-H, HO-EO₁₀₄PO₃₉EO₁₀₄-H, and HO-EO₂₇PO₆₁EO₂₇-H, wherein
EO=ethylene oxide and
PO=propylene oxide.

56-60. (canceled)

61. The method according to claim 48, wherein the biocompatible oil is ethylbutyrate in a concentration of about 20 mM to about 250 mM, the salt of a fatty acid is sodium caprylate in a concentration of about 10 mM to about 190 mM, the at least two emulsifiers other than the salt of a fatty acid in a concentration of about 4 mM to about 12 mM, and the water is normal saline.

62-63. (canceled)
